(19) United States
(12) Patent Application Publication
(10) Pub. No.: US 2023/0146429 A1
(43) Pub. Date: May 11, 2023

(54) BIOMETRIC AUTHENTICATION IN OPHTHALMIC DEVICES

(71) Applicant: Twenty Twenty Therapeutics LLC, South San Francisco, CA (US)

(72) Inventors: Hojr Sedaghat Pisheh, South San Francisco, CA (US); Dimitri Azar, South San Francisco, CA (US); Supriyo Sinha, Menlo Park, CA (US)

(21) Appl. No.: 18/051,593

(22) Filed: Nov. 1, 2022

Related U.S. Application Data
(60) Provisional application No. 63/277,460, filed on Nov. 9, 2021.

(51) Int. Cl. G06V 40/18; G06V 10/44; G06V 40/10; A61B 3/14; A61B 3/16; A61B 3/12; A61B 3/107; A61B 3/00; A61B 5/1171; A61B 3/18

(52) U.S. Cl. CPC G06V 40/197; G06V 10/44; G06V 40/193; G06V 40/15; A61B 3/14; A61B 3/165; A61B 3/1216; A61B 3/107; A61B 3/0008; A61B 5/1171; A61B 3/18

(57) ABSTRACT

Systems and methods for biometric authentication in ophthalmic devices are provided. In one embodiment, a system includes: an ophthalmic diagnostic device configured to obtain diagnostic measurements of a patient's eye, the patient's eye including an iris and a cornea; an imaging device configured to capture an image of the iris; an optical sensor integrated with the imaging module and configured to detect a position of the iris with respect to the optical sensor; and a computing device in communication with the ophthalmic diagnostic device, imaging device, and the optical sensor. The computing device may be configured to: determine, based on the image of the iris and the position of the iris, whether the iris matches a known patient's iris; and perform a diagnostic measurement of the patient's eye based on the determining.

### CROSS-REFERENCE TO RELATED APPLICATIONS

The present application claims the benefit of and priority to U.S. Provisional Patent Application No. 63/277,460, filed Nov. 9, 2021, the entirety of which is incorporated by reference herein.

### TECHNICAL FIELD

The present disclosure relates generally to devices and methods for performing biometric authentication of human eyes using ophthalmic devices, such as devices configured to measure intraocular pressure (TOP).

### BACKGROUND

Ophthalmic systems such as those designed for diagnosing or treating ocular diseases are typically shared by multiple patients. In some instances, a patient may forget to load, or incorrectly load, his/her profile in the device, resulting in the testing and/or treatment results not processed or implemented correctly. If the patient's data is being collected for machine learning or analytics, errors in enrollment would also corrupt the dataset for training and analysis. In some instances, ill-intentioned individuals may impersonate other patients, compromising security of any medical records and/or personal information safeguarded by the systems. For at least these reasons, a biometrics-based authentication process associated with using these ophthalmic systems (or other systems shared by multiple patients or users) is desired.

Existing authentication technologies have relied on biometrics such as iris, fingerprints, facial features, and/or other suitable human features. Of these common biometric recognition regimes, iris recognition has been widely used due to its exceptional power to avoid false matches across a large sample population. The iris of a human eye has a fine and complex texture that is determined randomly during embryonic gestation. Such complexity combined with its random nature significantly lowers the chance that two individuals may share the same iris texture. Even genetically identical individuals, as well as the left and the right eyes of the same individual, have completely different iris textures that can be observed by iris imaging. While existing authentication processes by iris recognition are generally adequate, they have not been entirely satisfactory in all aspects. In some instances, absent a robust data discrimination algorithm, the system may fail to correlate an existing patient's iris code (which is derived from the patient's iris image) with his/her profile in the database, leading to the patient's test and/or treatment results being falsely rejected (i.e., a false-negative authentication result). In some instances, falsely accepted (i.e., false-positive) authentication results may also be possible when ill-intentioned individuals impersonate biometric data of existing patients, thereby compromising the security of the patient's personal information. Therefore, for at least these reasons, improvements in the performance of authentication processes by iris recognition are desired.

### SUMMARY

According to one embodiment of the present disclosure, a system includes: an ophthalmic diagnostic device configured to obtain diagnostic measurements of a patient's eye, the patient's eye including an iris and a cornea; an imaging device configured to capture an image of the iris; an optical sensor integrated with the imaging module and configured to detect a position of the iris with respect to the optical sensor; and a computing device in communication with the ophthalmic diagnostic device, the imaging device, and the optical sensor, wherein the computing device is configured to: determine, based on the image of the iris and the position of the iris, whether the iris matches a known patient's iris; and perform a diagnostic measurement of the patient's eye based on the determining.

In some aspects, the ophthalmic diagnostic device is configured to measure intraocular pressure of the patient's eye. In some aspects, the system further includes an optical sensor configured to measure deflection of the patient's eye in response to pressure applied by the ophthalmic diagnostic device. In some aspects, the computing device is configured to determine deflection of the patient's eye in response to pressure applied by the ophthalmic diagnostic device using data obtained from the imaging device. In some aspects, the imaging device includes: a light source configured to illuminate the patient's eye with an incident light; and a camera configured to capture an image of the patient's eye that includes a reflection of the incident light. In some aspects, the imaging device is configured to detect a Purkinje image produced by the incident light reflected from the cornea of the patient's eye. In some aspects, the computing device is configured to calculate a curvature of the cornea based on the Purkinje image.

According to another embodiment of the present disclosure, a system includes: an iris imaging module including: a light source configured to illuminate iris of an eye with an incident light; and a camera configured to capture an image of the iris, the image including a reflection of the incident light on the eye; a proximity sensor in communication with the iris imaging module and configured to measure a size of the iris; and an iris authentication module including a memory and a processor in communication with the memory, wherein the iris authentication module is configured to: determine, based on the reflection of the incident light on the eye in the image, a curvature of the eye, and authenticate the iris based on the curvature of the eye.

In some aspects, the light source includes at least one light-emitting diode (LED). In some aspects, the system further includes an ophthalmic device integrated with at least one of the iris imaging module and the proximity sensor. In some aspects, the ophthalmic device is a non-contact tonometer. In some aspects, the ophthalmic device is an ocular drug delivery system. In some aspects, the light source is configured to emit the incident light in a defined pattern, and the image detected by the camera is a Purkinje image including a distorted reflection of the defined pattern off the cornea.

According to another embodiment of the present disclosure, a method includes: providing an iris recognition system including at least an imaging module and a proximity sensor; capturing an image of a patient's iris using the imaging module; determining a distance of the iris from the proximity sensor; determining a size of the based on the determined distance and the captured image; determining, based on the captured image, a curvature of the patient's cornea; and comparing the captured image and at least one of the size of the iris or the curvature of the cornea with corresponding data stored in a database to confirm an identity of the patient, wherein the confirmation is based on a matching threshold.

In some aspects, the method further includes: providing an ophthalmic system configured to obtain a diagnostic measurement of the patient's eye, wherein the ophthalmic system is integrated with the iris recognition system; and obtaining, based on confirming the identity of the patient, the diagnostic measurement of the patient's eye using the ophthalmic system. In some aspects, the method further includes adjusting the matching threshold based on confirming the identity of the patient. In some aspects, the ophthalmic system includes a non-contact tonometer. In some aspects, the determining the curvature of the cornea includes analyzing a Purkinje image reflected from the cornea. In some aspects, the determining the size of the iris includes: calculating a relative dimension of the iris based on the captured image; determining a position of the iris with respect to the proximity sensor; and determining an absolute dimension of the iris based on the calculated relative dimension and the position of the iris. In some aspects, the matching threshold is a Hamming distance threshold, and the comparing the captured image and the at least one of the size of the iris or the curvature of the cornea includes comparing a difference between the captured image and a stored authentication image to the Hamming distance threshold.

This summary is provided to introduce a selection of concepts in a simplified form that are further described below in the detailed description. This summary is not intended to identify key features or essential features of the claimed subject matter, nor is it intended to limit the scope of the claimed subject matter. A more extensive presentation of features, details, utilities, and advantages of the jet pump for noncontact tonometry, as defined in the claims, is provided in the following written description of various embodiments of the disclosure and illustrated in the accompanying drawings.

### BRIEF DESCRIPTION OF THE DRAWINGS

The present disclosure is best understood from the following detailed description when read with the accompanying figures. It is emphasized that, in accordance with the standard practice in the industry, various features are not drawn to scale and are used for illustration purposes only. In fact, the dimensions of the various features may be arbitrarily increased or reduced for clarity of discussion.

FIG. 1 is a diagrammatic view of an ophthalmic system, according to at least one embodiment of the present disclosure.

FIG. 2 is a functional block diagram of the ophthalmic system, in portion or in entirety, as depicted in FIG. 1, according to at least one embodiment of the present disclosure.

FIG. 3 is a schematic view of the ophthalmic system, in portion or in entirety, as depicted in FIG. 2, according to at least one embodiment of the present disclosure.

FIGS. 4A, 4B, 4C, and 4D each illustrate a schematic view of the ophthalmic system, in portion or in entirety, as depicted in FIG. 3, according to at least one embodiment of the present disclosure.

FIG. 5 is a schematic view of a portion of a human eye interacting with an incident light, according to at least one embodiment of the present disclosure.

FIG. 6 is a schematic view of a portion of a human eye interacting with an incident light, according to at least one embodiment of the present disclosure.

FIGS. 7A and 7B illustrate a flowchart of an example method for using the ophthalmic system as depicted in one or more of the FIGS. 1-4D, according to at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the present disclosure is described in terms of devices and systems configured to measure TOP or to deliver ocular drugs to a human eye, it is understood that the disclosure is not intended to be limited to these applications. The devices and systems are equally well suited to any application having an operation interface shared by multiple patients whose data are stored and processed in a common database. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Ophthalmic systems designed to diagnose or treat ocular diseases are typically utilized by multiple patients in settings such as a hospital or a doctor's office. A database of information is accessed each time before a patient, existing or new, is subject to testing and/or treatment by such systems, and the corresponding testing and/or treatment results are processed and stored in the database for further medical assessment. Accordingly, for purposes of safeguarding patients' personal information (including, but not limited to, medical records) and implementing appropriate testing and/or treatments, a robust and secure authentication system is desired.

The present disclosure provides systems, and methods of using the same, for performing a biometric authentication process that utilizes a series of iris recognition and matching processes. In the present embodiments, aspects of the patient's ocular metrics including, for example, the size of the iris and the curvature of the cornea, obtained from imaging the patient's eye are employed to confirm the patient's identity during the authentication process. When such a combination of metrics is collected and analyzed, an algorithm of the authentication process may be adjusted to accommodate possible variations in imaging conditions for purposes of reducing false rejection rate (FRR). Furthermore, information deduced from the combined metrics may also safeguard against illicit access of the database to reduce false acceptance rate (FAR).

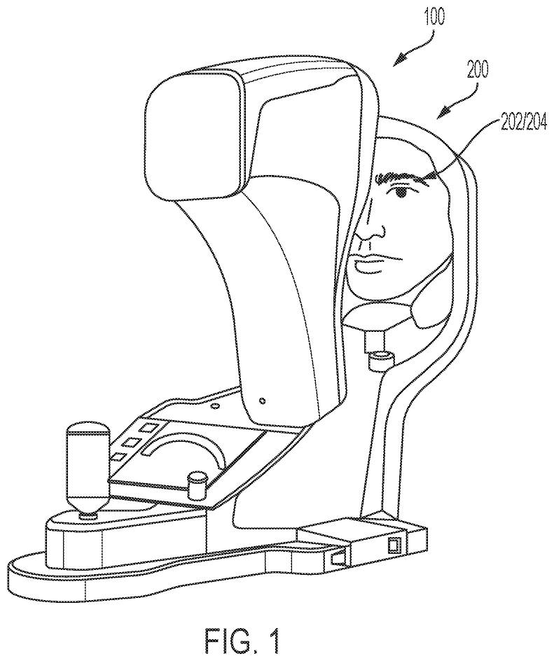

FIG. 1 illustrates a schematic of using an ophthalmic system (hereafter referred to simply as system) 100 to treat a patient 200's eye 202 according to various embodiments of the present disclosure. In the depicted embodiments, the system 100 is a non-contact tonometer designed to measure the IOP of each patient's eye(s). In other embodiments, the system 100 may include an automated ocular drug delivery system designed to dispense a drug according to each patient's personalized dosage and frequency. In some embodiments, the system 100 is configured to store and process multiple patients' information. In some embodiments, the system 100 may be configured to store and process IOP data and associate the data with the corresponding eye of one or more patients. For example, the system 100 may include a memory configured to store a plurality of IOP measurements at various points in time. The system 100 may be further configured to connect with a network, such as a local area network (LAN), and wide area network (WAN), or any other suitable network. In some aspects, the system 100 is configured to communicate ophthalmic diagnostic data or measurements to a remote server, which can be accessed by the patient's physician. In some embodiments, the system 100 provides data safety features for a device that requires biometric authentication to function properly, such as a personal mobile device. It is noted that while the following description and the accompanying figures are directed to the system 100 being configured to perform non-contact tonometry, the present embodiments are equally suitable for other applications. Furthermore, although FIG. 1 depicts the system 100 in a stationary, table-top configuration, embodiments of the present disclosure are equally applicable for portable, hand-held devices. In some embodiments, the patient 200's eyes are tested one at a time, such that the IOP data specific to each eye is recorded separately in a database stored in a computing device, such as a computer.

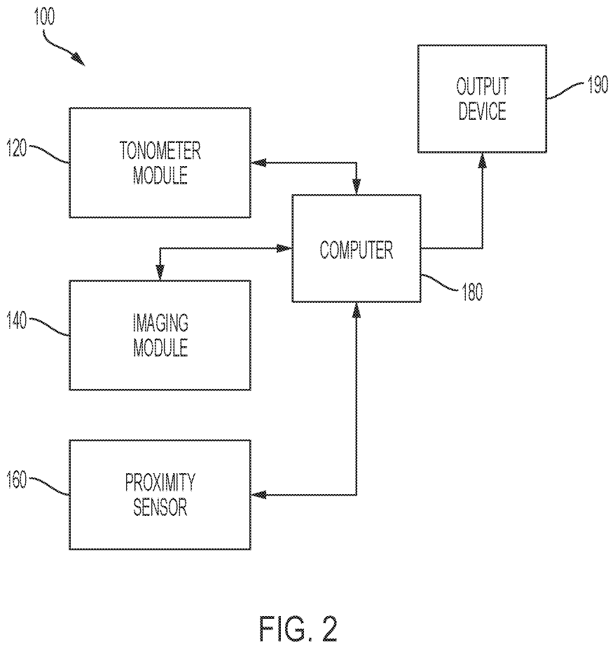

FIG. 2 is a diagrammatic view of the system 100. The system 100 may include a tonometer module 120, an imaging module 140, and a proximity sensor 160, all of which are in communication with a computer 180 that is further connected to an output device 190. In some embodiments, the tonometer module 120 includes devices configured to deliver a known amount of pressure to a patient's cornea and devices configured to measure deflection of the cornea in response to the applied pressure. In further embodiments, the imaging module 140 includes a plurality of light-emitting and light-sensing devices for illuminating and capturing images of the patient's eyes. Details of the various modules of the system 100 are discussed below.

In some embodiments, modules of the system 100 can be controlled independently by the computer 180. In this regard, these modules may each include one or more user input devices not separately depicted in FIG. 2. In some embodiments, as discussed in detail below, these modules complement each other to complete the biometric authentication process. In some embodiments, these modules share one or more components in common to form an integrated system. For embodiments in which the system 100 performs functions other than measuring a patient's IOP, the tonometer module 120 may be replaced by other functional modules accordingly. For example, if the system 100 is configured to deliver an ocular drug, then the tonometer module 120 may be replaced by a dispensing module instead. Though not depicted, the system 100 may further include other components, such as power supply and user input device(s).

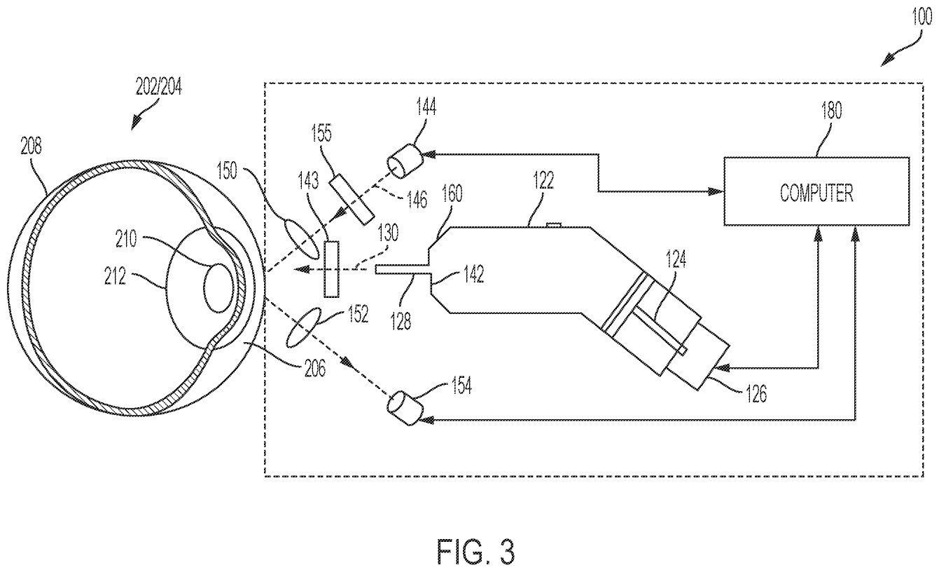

Generally, the tonometer module 120 may be configured to release a puff of air (e.g., air puff 130 depicted in FIG. 3) at the cornea (e.g., cornea 206 as depicted in FIG. 3) of the eye 202 (or 204) as in the case of non-contact tonometry, or to directly apply pressure to the cornea using a small probe (e.g., a piston), as in the case of contact (or applanation) tonometry, and subsequently measure the deflection of the cornea of the eye 202 in response to the puff of air. The air pressure required to temporarily flatten a region of the cornea is equal to the IOP of the eye 202. In some instances, the IOP of the patient 200's two eyes may differ. Accordingly, it may be beneficial to authenticate IOP data obtained for each eye, so that the data can be stored and/or analyzed separately. The imaging module 140 is configured to capture an image of the patient 200's iris (e.g., iris 212 as depicted in FIG. 3) and transmits the image to the computer 180, which subsequently processes and converts the image into a bit pattern encoding information of the iris for comparison against an existing iris code in a database stored in the computer 180. Operating in conjunction with the imaging module 140, the proximity sensor 160 can determine an axial position of the eye relative to the system 100, which then may be used to determine, based on the captured iris image, the size of the iris to scale.

The computer 180 is operable to control one or more of the modules of the system 100, store the information collected by the modules, and process such information for further assessment. For example, the computer 180 is configured to receive image data obtained from the imaging module 140 and/or the proximity sensor 160 and extrapolate and/or analyze metrics such as the a size of the iris, the deflection of the cornea, one or more Purkinje images, or other suitable metrics. The computer 180 may include one or more clock, memory, logic, or processing devices. Logic or processing devices may be application-specific or for general purpose. The computer 180 may employ any combination of hardware, software, and firmware to perform its functions. The computer 180 may further employ a fixed instruction set provided in read-only memory (ROM) or could have an updatable instruction set provided in programmable read-only memory (PROM), electrically erasable programmable read-only memory, flash memory, or any equivalent thereof. The computer 180 may be configured to communicate with a remote server, or a local device such as a laptop, tablet, or handheld device. In the present embodiments, the database containing registered iris codes that belong to multiple patients is accessed through the computer 180 to provide biometric authentication of a patient's identity. The authentication results, as well as the IOP measurements, may be displayed and/or reported to an authorized user (e.g., the patient 200 and/or a healthcare provider) via the output device 190.

Referring to FIG. 3, a portion of the system 100 configured as the tonometer module 120 includes at least a chamber 122 that surrounds a piston pump 124 operatively connected to a motor 126, which causes the piston pump 124 to force air through the chamber 122, forming an air puff 130 through a nozzle 128 to strike the eye 202. In some embodiments, the driving force for forming the air puff 130 is proportional to a current applied to the motor 126 and may be initiated and controlled by the computer 180. The chamber 122 may be a portion of a stationary system or a hand-held system configured to measure the TOP of the patient 200. In some embodiments, the tonometer module 120 optionally includes an optical system for measuring the deflection of the cornea 206.

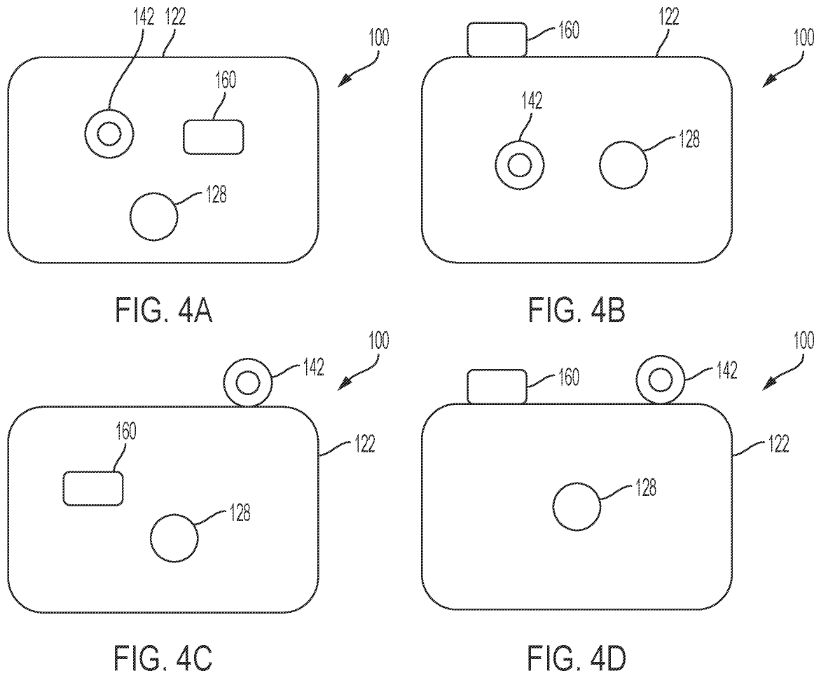

In the present embodiments, referring to FIG. 3 and FIGS. 4A-4D collectively, a portion of the system 100 configured as the imaging module 140 includes at least an image capturing device (ICD) 142, such as a camera, positioned to face the eye 202. The ICD 142 is configured to capture an image of the iris 212 illuminated by a light source 144 disposed at angle relative to the eye 202. The ICD 142 may be any suitable device capable of capturing an image of the iris 212 at a resolution acceptable for generating an iris code for biometric authentication. For example, the ICD 142 may include a charge-coupled device (CCD), a complementary metal-oxide semiconductor (CMOS) imaging device, or any other suitable imaging device. The captured iris image may be subsequently transmitted to the computer 180 for further analysis as discussed in detail below. In some embodiments, the ICD 142 is configured to measure the deflection of the cornea 206 when measuring the IOP. In some embodiments, a spectral filter 143 is disposed in front of the ICD 142 to remove light having wavelengths that may cause specular reflections of the eye 202.

The light source 144 emits an incident light 146 toward the eye 202. The light source 144 is configured to illuminate the eye 202 in any suitable wavelength range, such as in the near infrared (IR) range (i.e., the incident light 146 having a wavelength in the range of about 700 nm to about 1100 nm) or in the visible light range (i.e., the incident light 146 having a wavelength in the range of about 400 nm to about 700 nm). In the present embodiments, the light source 144 illuminates the eye 202 for purposes of capturing an image of the iris 212 and/or producing one or more Purkinje images as discussed in detail below. In some embodiments, the light source 144 is a light-emitting diode (LED). In some embodiments, though not depicted, the system 100 includes more than one light source 144 disposed at different angles relative to the eye 202. For example, a first light source 144 may be configured to illuminate the iris 212, while a second light source 144 may be configured to illuminate the eye 202 to obtain one or more Purkinje images. In some embodiments, a filter (not depicted) may be placed in front of the light source 144 to reduce the optical power of the incident light 146 for the safety of the eye 202.

After impinging upon the eye 202, the incident light 146 is reflected through a lens 152 and detected by an optical sensor 154. When used as a component of the tonometer module 120 according to some embodiments, the optical sensor 154 may facilitate the measurement of the deflection of the cornea 206. In the present embodiments, the ICD 142, the light source 144, the optical sensor 154, the lenses 150 and 152, the spectral filter 143 and/or other components of the system 100 are collectively configured to enhance iris features not distinguishable in visible light to naked eyes and to remove specular reflections that would otherwise obscure such iris features in the captured iris image.

In the present embodiments, the image module 140 further includes an image projector 155 that operates in conjunction with the light source 144 to project an image onto various optical planes of the eye 202, resulting in one or more Purkinje images to be captured by the ICD 142 and subsequently transmitted to the computer 180 for analysis. The image projector 155 may produce a pattern including, for example, a line-grid pattern, a dot grid, crosshairs, concentric rectangles, concentric circles, or other suitable patterns. In an example embodiment, the image projector 155 is a dot projector. In some embodiments, referring to FIG. 3, the image projector 155 is disposed between the light source 144 and the eye 202. In some embodiments, the image projector 155 is integrated with the light source 144.

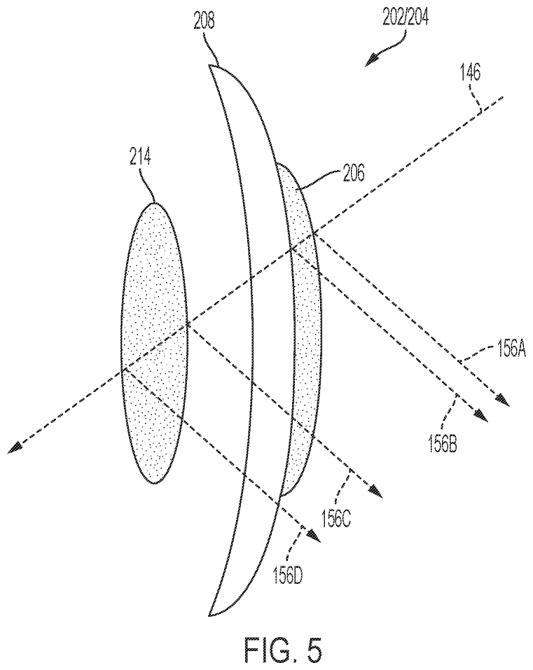

FIG. 5 schematically depicts four Purkinje images 156A-156D produced by the eye 202 when illuminated by the incident light 146. Purkinje images of an object are typically formed at various optical planes in an eye and may be used to analyze various features of the eye, such as the curvature of the cornea, based on their respective characteristics. Generally, the first Purkinje image 156A is an image reflected from the anterior surface of the cornea 206; the second Purkinje image 156B is an image reflected from the posterior surface of the cornea 206; the third Purkinje image 156C is an image reflected from the anterior surface of lens 214; and the fourth Purkinje image 156D is an image reflected from the posterior surface of the lens 214. In some embodiments, the first Purkinje image 156A may be captured by the ICD 142 and processed by the computer 180 to extrapolate the curvature (defined by a radius, for example) of the cornea 206.

Referring back to FIG. 3 and FIGS. 4A-4D, the system 100 further includes the proximity sensor 160. The proximity sensor 160 may be coupled to, or integrated with, the tonometer module 120 and the imaging module 140. In the present embodiments, the proximity sensor 160 may be used in tandem with the imaging module 140 to obtain an actual (i.e., true-to-scale) size of the iris 212 in terms of radius, diameter, and/or area based on the captured iris image, which may describe the size of the iris 212 in terms of pixels. In a non-telecentric imaging system, such as that provided by the system 100, the size of the iris 212 varies with the distance at which the iris image is captured. In some examples, the diameter of a patient's iris may be about 11 mm to about 13 mm. In this regard, the actual size of the iris 212 may be deduced by measuring a separation distance along an axis between the iris 212 and the proximity sensor 160. In some embodiments, the proximity sensor 160 is positioned on or near the same imaging plane as the ICD 142 to ensure accurate determination of such separation distance. In some embodiments, the proximity sensor 160 is placed along the optical axis of the ICD 142 with a known separation distance. The proximity sensor 160 may be any suitable optical sensor capable of detecting an axial position of the iris 212. In the present embodiments, the proximity sensor 160 may be configured with a resolution on the order of a millimeter (mm), with sub-millimeter resolution, or with any suitable resolution.

In some embodiments, the proximity sensor 160 includes a source (not depicted separately) configured to emit radiation (e.g., electromagnetic radiation) and a detector (not depicted separately) configured to sense changes in the emitted radiation as a return signal, where such changes are processed by the computer 180 to obtain the axial separation distance between the iris 212 and the proximity sensor 160. Subsequently, correlating such separation distance with the measured size of the iris 212, which is calculated based on the captured iris image, provides the actual size of the iris 212. In the present embodiments, the size of the iris 212 is equivalent to an area between an outer perimeter of the iris 212 defined by sclera 208 and an inner perimeter of the iris 212 defined by pupil 210.

It is noted that the present disclosure does not limit the physical arrangement of the various components of the system 100 to any specific configuration. For example, the nozzle 128, the ICD 142, and the proximity sensor 160 may be arranged on the same frontal surface of the chamber 122 that is opposite to the eye 202, as depicted in FIG. 4A. Alternatively, as depicted in FIGS. 4B-4D, one or both of the ICD 142 and the proximity sensor 160 may be disposed on a top surface of the chamber 122, though they remain along the same imaging plane.

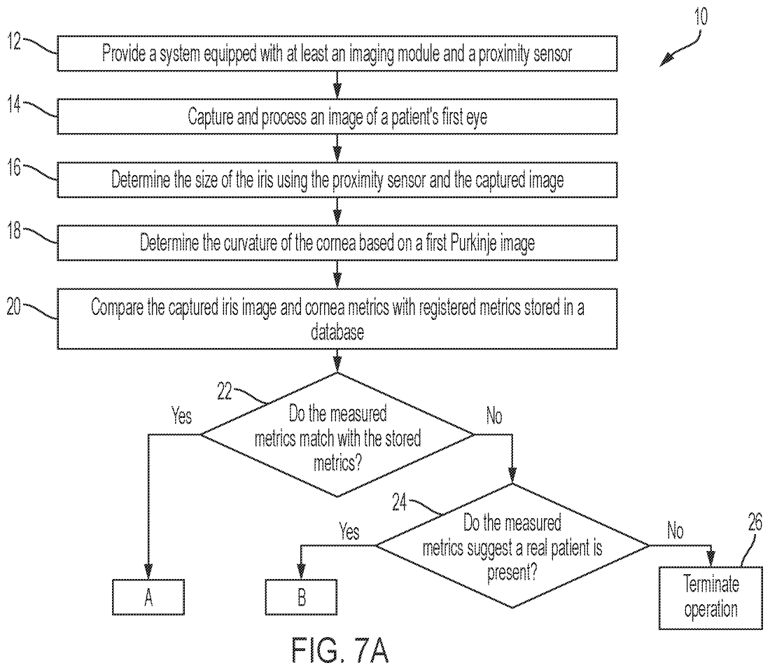

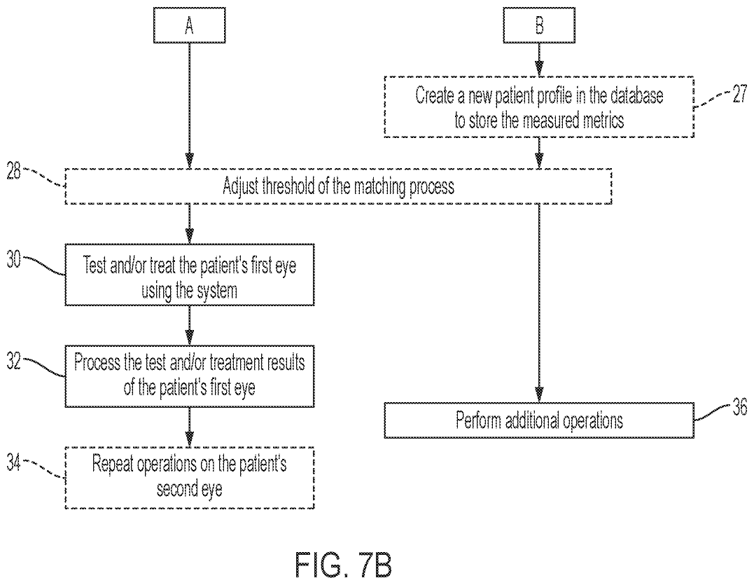

Referring now to FIGS. 7A and 7B collectively, a flowchart of a method 10 of using the system 100 is illustrated according to various aspects of the present disclosure. Method 10 is merely an example and is not intended to limit the present disclosure beyond what is explicitly recited in the claims. Additional operations can be provided before, during, and after method 10, and some operations described can be replaced, eliminated, or moved around for additional embodiments of the method.

Referring to FIG. 7A, method 10 at operation 12 provides the system 100 as discussed in detail above with respect to FIGS. 1-4D. The system 100 may be non-contact tonometry system equipped with the tonometer module 120, the imaging module 140, and the proximity sensor 160 in communication with the computer 180, which may be further connected to the output device 190. In some embodiments, instead of measuring the patient 200's IOP, the system 100 may be configured to deliver an ocular drug to a patient and further equipped with the imaging module 140 and the proximity sensor 160, all of which are in communication with the computer 180. In some embodiments, the system 100 may be integrated into a device that provides personal data safety features, such as a personal mobile device.

Subsequently, method 10 at operation 14 captures an image of the patient 200's first eye 202 using the imaging module 140. In the present embodiments, the imaging module 140 is configured to illuminate the iris 212 of the first eye 202 with the incident light 146 provided by the light source 144, such as an LED. In some embodiments, the incident light 146 has a wavelength in the IR range. In some embodiments, the spectral filter 143 is used to remove any ambient visible light, thereby enhancing relevant features of the captured iris image. In the present embodiments, the captured iris image is processed by the computer 180 to form a captured iris code (e.g., a bit pattern) that encompasses various features, such as color, texture, and size, of the iris 212. In some embodiments, the computer 180 executes algorithms to remove any objects (e.g., eyelashes and eyelids) and/or specular reflections before converting the captured iris image into the captured iris code.

At operation 16, method 10 determines the actual size of the iris 212 using the captured iris image and the proximity sensor 160, which determines an axial position of the iris 212 relative to the proximity sensor 160. The actual size of the iris 212 may be determined based on the principle that the measured size of the iris 212 (encoded in the captured iris image) is proportional to the separation distance between the iris 212 and the proximity sensor 160 in a non-telecentric imaging system. In some examples, the measured size of the iris 212 may be obtained in units of pixels, while the actual size of the iris 212 may be presented in units of mm, inches, and/or mm², for example.

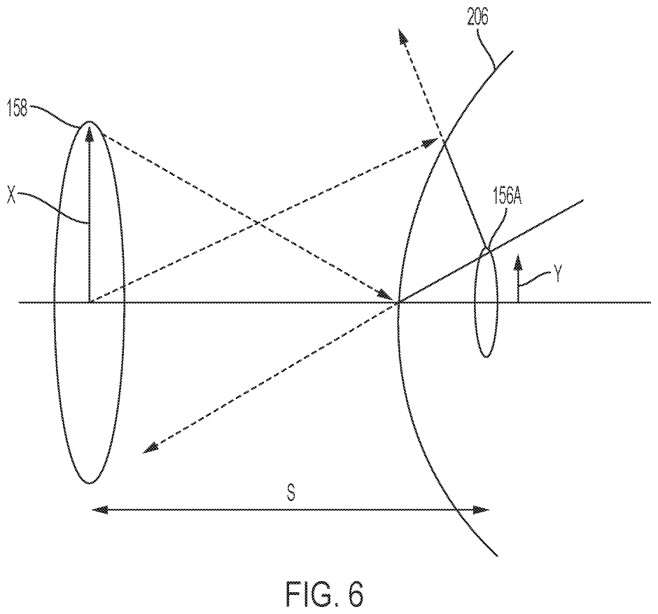

At operation 18, method 10 determines the curvature of the cornea 206 of the first eye 202. As discussed in detail above, the first Purkinje image 156A produced by the first eye 202 may be used to determine the curvature of the cornea 206, a method termed keratometry. FIG. 6 schematically illustrates an example keratometry process, which includes placing an object 158 defined by a height X in front of the cornea 206, the object 158 being illuminated by the light source 144 (not depicted) of the imaging module 140. The first Purkinje image 156A of the object 158 is formed behind the cornea 206 (a virtual image is shown here) and defined by a height Y at a distance S away from the object 158. The radius R of the cornea 206 may then be calculated by using the following equation:

$$R = \frac{2 SY}{X} .$$

Additionally or alternatively, a fixed pattern of LED illumination may be used to infer the curvature of the cornea 206. For example, an array of LEDs could be fixed in a grid pattern and the corresponding Purkinje image(s) may be used to infer not only the curvature but also the topography of the cornea 206.

In the present embodiments, an accurate measurement of the curvature of the cornea 206 is used in conjunction with the captured iris image and the actual size of the iris 212 to ascertain that the first eye 202 being authenticated is indeed from a real patient rather than a reproduced image of the patient's eye. Specifically, if a reproduced image of the patient's eye is presented before the system 100, it is probable that some aspects of the captured iris code may be consistent with a registered iris code for the patient stored in the database; however, the actual curvature of the cornea 206 obtained using the first Purkinje image 156A would not represent a meaning value, i.e., the radius R may be infinite.

The present embodiments do not limit the order in which operations 16 and 18 are implemented. For example, operation 16 may be implemented before operation 18, or vice versa. Alternatively, operations 16 and 18 may be implemented simultaneously.

Thereafter, method 10 at operation 20 compares the measured iris and cornea metrics, including the iris code, the actual size of the iris 212, and the curvature of the cornea 206, with registered metrics stored in the database of the computer 180. In some embodiments, the computer 180 compares the captured iris code against each registered iris code in the database on a bit-by-bit basis. A positive matching result occurs when at least a specific fraction of the total bits match, thereby satisfying a Hamming distance threshold predetermined for such a matching (i.e., authentication) process. In the present embodiments, the Hamming distance threshold (hereafter referred to as the threshold) is a concept commonly understood by persons ordinarily skilled in the art. By way of example, the threshold for the matching process discussed herein may be about 0.3. Typically, increasing the threshold increases the FAR (i.e., the false-positive results) of the matching process and decreases the FRR (i.e., the false-negative results), and decreasing the threshold decreases the FAR and increases the FRR. Advantageously, the present embodiments provide a system and a method of using the same for increasing the threshold to bring about a reduced FRR while also lowering the FAR, such that both the robustness and the safety features of the authentication process may be improved.

In the present embodiments, besides comparing the bit pattern of the captured iris code with those of the registered iris codes, method 10 at operation 20 may including comparing the actual size of the iris 212 obtained from the measurements of the proximity sensor 160 with actual sizes of irises stored in the database to further ascertain the identity of the patient 200. Advantages of this additional process of comparison, which increases the threshold, are at least two-fold. Firstly, the increased threshold would improve immunity of the authentication process against unfavorable ambient lighting conditions, focus blur associated with the captured iris image, angle dependence of the captured iris image, and/or other conditions that would otherwise produce false-negative authentication results, leading to a more robust and user-friendly authentication process. Secondly, the increased threshold would require any person pretending to be the patient 200 or presenting a reproduced image of the patient 200's first eye 202 to match the actual, true-to-scale size of the iris 212 with a high degree of accuracy, thereby improving the security of the authentication process. Alternatively or additionally, method 10 at operation 20 compares the curvature of the cornea 206 obtained from the first Purkinje image 156A with values of corneal curvature stored in the database to raise the threshold and obtain similar advantages.

Subsequently, method 10 at operation 22 evaluates whether the measured metrics match the registered metrics according to the predetermined threshold. If a match has occurred, method 10 proceeds to operation 28 as depicted in FIG. 7B; otherwise, method 10 proceeds to operation 24 to determine whether the measured metrics, such as the curvature of the cornea 206 determined by the first Purkinje image 156A, suggest a real patient is present. If such a condition is met, i.e., the measured curvature of the cornea 206 is a value typical of a human cornea, then method 10 proceeds to operation 27 as depicted in FIG. 7B. If such a condition is not met, (i.e., the measured curvature of the cornea 206 is not a value typical of a human cornea, then method 10 is terminated at operation 26. Scenarios leading to termination of operation may include, for example, if the measured curvature of the cornea 206 is infinite, which suggests that a flat substrate (e.g., an image of a human cornea printed on a piece of paper), rather than a curved object (e.g., an actual human cornea), is present. In some embodiments, the actual size of the iris 212 may also be used to determine whether a real patient is present, since the size of the iris on a printed image must closely match that of an actual iris to scale, which may be difficult to achieve.

Referring now to FIG. 7B, on condition that the measured curvature of the cornea 206 is a value typical of a human cornea, method 10 may proceed to operation 27 by creating a new patient profile in the database to store the measured metrics. This operation may be optional and may be omitted according to protocols set forth by an authorized user of the system 100.

On condition that the measured metrics match with registered metrics in the database, e.g., a positive match has occurred, or after confirming a new patient is present, method 10 may proceed to operation 28 to adjust the threshold of the matching process implemented at operations 20 and 22. In some embodiments, the threshold of the matching process may be increased such that incorporating the actual size of the iris 212 and/or the curvature of the cornea 206 into the conventional iris code matching process results in a lowered FRR and thus a more robust authentication outcome. Advantageously, if the size of the iris 212 and/or the curvature of the cornea 206 of a given patient is an outlier, then adjusting the threshold at operation 28 allows such patient to be authenticated with greater accuracy in the future. In some embodiments, the degree by which the threshold is increased is determined through simulation and experimentation for the system 100 under typical testing conditions that incorporate the measurements of the various iris and cornea metrics discussed herein. Unless desired by an authorized user of the system 100, operation 28 may be implemented a single time after a successful match for a given patient is completed, such that the adjusted threshold is kept constant for subsequent authentication processes. In other embodiments, operation 28 may be implemented multiple times after a successful match. In some embodiments, operation 28 may be omitted, i.e., the threshold remains unchanged for the patient 200 (as well as other patients using the system 100), such that incorporating the actual size of the iris 212 and/or the curvature of the cornea 206 into the conventional iris code matching process leads to a reduced FAR, thereby improving the data security associated with the authentication process. In this regard, obtaining the additional metrics improves the overall efficacy of the authentication process and further allows the authentication process to be tailored to a greater number of users by incorporating customizable comparison parameters.

If the system 100 functions as an ophthalmic system, such as one configured to perform non-contact tonometry or to deliver an ocular drug, method 10 then proceeds to operation 30 and performs any relevant test and/or treatment on the first eye 202 of the patient 200. Subsequently, method 10 at operation 32 processes any data associated with such test and/or treatment and stores them in the database for record-keeping and/or further assessment.

With respect to using the system 100 to measure a patient's TOP during non-contact tonometry, the present embodiments allow the patient's two eyes to be identified and tested separately, such that the TOP of each eye may be recorded correctly and categorized automatically. In addition, if the authorized user fails to first load the patient's profile, i.e., bypassing the authentication process discussed above with respect to operations 14-24, the system 100 will alert the authorized user to do so first before proceeding to the TOP measurements. With respect to using the system 100 to deliver an ocular drug, the present embodiments ensure that the correct dosage of the correct ocular drug is delivered to each eye of a patient at a prescribed time and frequency, thereby improving the accuracy of the drug delivery process.

Thereafter, method 10 at operation 34 may proceed to repeating operations 14-32 on the second eye 204 of the patient 200 if such test and/or treatment is necessary. In some embodiments, operations 30-34 are optional and may be omitted if the system 100 does not perform the functions of an ophthalmic system. In some embodiments, after adjusting the threshold of the matching process at operation 28, method 10 may proceed directly to operation 36 and perform additional operations relevant to the functions of the system 100. For instances, if the system 100 is integrated with a device that requires biometric authentication to gain access, then after adjusting the threshold of the matching process at operation 28, method 10 at operation 36 may allow the authorized user to unlock the device and perform additional operations.

Communication (including but not limited to software updates, firmware updates, or readings from the device) to and from the system 100 could be accomplished using any suitable wireless or wired communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches.

The logical operations making up the embodiments of the technology described herein may be referred to variously as operations, steps, objects, elements, components, or modules. It should be understood that these may be performed or arranged in any order, unless explicitly claimed otherwise or a specific order is inherently necessitated by the claim language. It should further be understood that the described technology may be employed as a standalone device or as a component of other devices.

All directional references, e.g., upper, lower, inner, outer, anterior, posterior, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the system 100. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

Furthermore, when a number or a range of numbers is described with “about,” “approximate,” and the like, the term is intended to encompass numbers that are within a reasonable range including the number described, such as within +/−10% of the number described or other values as understood by person skilled in the art. For example, the term “about 5 nm” encompasses the dimension range from 4.5 nm to 5.5 nm. Still further, the present disclosure may repeat reference numerals and/or letters in the various examples. This repetition is for the purpose of simplicity and clarity and does not in itself dictate a relationship between the various embodiments and/or configurations discussed.

The above specification, examples and data provide a complete description of the structure and use of exemplary embodiments of the jet pump for noncontact tonometry as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter. For example, the jet pump could be used to produce controlled puffs of other gases than ambient air, including but not limited to oxygen, nitrogen, helium, and argon, or of gases that contain colorants, odorants, medications, or other materials. Additionally, some or all of the components of the jet pump may be contained within a housing, either alone or with other components such as a battery and/or power supply.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims. Persons skilled in the art will recognize that the devices, systems, and methods described above can be modified in various ways not explicitly described or suggested above. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
an ophthalmic diagnostic device configured to obtain diagnostic measurements of a patient's eye, the patient's eye including an iris and a cornea;
an imaging device configured to capture an image of the iris;
an optical sensor integrated with the imaging device and configured to detect a position of the iris with respect to the optical sensor; and
a computing device in communication with the ophthalmic diagnostic device, the imaging device, and the optical sensor, wherein the computing device is configured to:
determine, based on the image of the iris and the position of the iris, whether the iris matches a known patient's iris; and
perform a diagnostic measurement of the patient's eye based on the determining.

2. The system of claim 1, wherein the ophthalmic diagnostic device is configured to measure intraocular pressure of the patient's eye.

3. The system of claim 2, further comprising an optical sensor configured to measure deflection of the patient's eye in response to pressure applied by the ophthalmic diagnostic device.

4. The system of claim 2, wherein the computing device is configured to determine deflection of the patient's eye in response to pressure applied by the ophthalmic diagnostic device using data obtained from the imaging device.

5. The system of claim 1, wherein the imaging device includes:
a light source configured to illuminate the patient's eye with an incident light; and
a camera configured to capture an image of the patient's eye that includes a reflection of the incident light.

6. The system of claim 5, wherein the imaging device is configured to detect a Purkinje image produced by the incident light reflected from the cornea of the patient's eye.

7. The system of claim 6, wherein the computing device is configured to calculate a curvature of the cornea based on the Purkinje image.

8. A system, comprising:
an iris imaging module including:
a light source configured to illuminate iris of an eye with an incident light; and
a camera configured to capture an image of the iris, the image including a reflection of the incident light on the eye;
a proximity sensor in communication with the iris imaging module and configured to measure a size of the iris; and
an iris authentication module including a memory and a processor in communication with the memory, wherein the iris authentication module is configured to:
determine, based on the reflection of the incident light on the eye in the image, a curvature of the eye, and
authenticate the iris based on the curvature of the eye.

9. The system of claim 8, wherein the light source includes at least one light-emitting diode (LED).

10. The system of claim 8, further comprising an ophthalmic device integrated with at least one of the iris imaging module and the proximity sensor.

11. The system of claim 10, wherein the ophthalmic device is a non-contact tonometer.

12. The system of claim 10, wherein the ophthalmic device is an ocular drug delivery system.

13. The system of claim 8, wherein the light source is configured to emit the incident light in a defined pattern, and wherein the image detected by the camera is a Purkinje image including a distorted reflection of the defined pattern off a cornea.

14. A method, comprising:
providing an iris recognition system including at least an imaging module and a proximity sensor;
capturing an image of a patient's iris using the imaging module;
determining a distance of the iris from the proximity sensor;
determining a size of the iris based on the determined distance and the captured image;
determining, based on the captured image, a curvature of a cornea of the patient; and
comparing the captured image and at least one of the size of the iris or the curvature of the cornea with corresponding data stored in a database to confirm an identity of the patient, wherein the confirmation is based on a matching threshold.

15. The method of claim 14, further comprising:
providing an ophthalmic system configured to obtain a diagnostic measurement of the patient's eye, wherein the ophthalmic system is integrated with the iris recognition system; and
obtaining, based on confirming the identity of the patient, the diagnostic measurement of the patient's eye using the ophthalmic system.

16. The method of claim 15, further comprising adjusting the matching threshold based on confirming the identity of the patient.

17. The method of claim 15, wherein the ophthalmic system includes a non-contact tonometer.

18. The method of claim 14, wherein the determining the curvature of the cornea includes analyzing a Purkinje image reflected from the cornea.

19. The method of claim 14, wherein the determining the size of the iris includes:
calculating a relative dimension of the iris based on the captured image;
determining a position of the iris with respect to the proximity sensor; and
determining an absolute dimension of the iris based on the calculated relative dimension and the position of the iris.

20. The method of claim 14, wherein the matching threshold is a Hamming distance threshold, and wherein the comparing the captured image and the at least one of the size of the iris or the curvature of the cornea includes comparing a difference between the captured image and a stored authentication image to the Hamming distance threshold.
